(19) United States
(12) Patent Application Publication
(10) Pub. No.: US 2022/0346677 A1
(43) Pub. Date: Nov. 3, 2022

(54) SYSTEMS AND METHODS FOR BLOOD GLUCOSE MANAGEMENT USING CONCENTRATED INSULIN

(71) Applicant: Pacific Diabetes Technologies Inc, Portland, OR (US)

(72) Inventors: William Kenneth Ward, Portland, OR (US); Robert S. Cargill, Portland, OR (US); Thomas L. Seidl, Wilsonville, OR (US); Matthew Breen, Beaverton, OR (US)

(21) Appl. No.: 17/730,741

(22) Filed: Apr. 27, 2022

Related U.S. Application Data
(60) Provisional application No. 63/181,083, filed on Apr. 28, 2021.

(51) Int. Cl. A61B 5/145

(52) U.S. Cl. CPC A61B 5/14532; A61B 5/14546; A61M 2230/201

(57) ABSTRACT

The present disclosure provides systems and methods for managing blood glucose in a subject. An insulin delivery cannula may comprise a hollow tube comprising a proximal end and a distal end, wherein the proximal end is in fluid communication with a source of a concentrated insulin or insulin analog formulation of at least about 150 units per milliliter. The distal end may be configured to deliver the concentrated insulin or insulin analog formulation into a subcutaneous space. A continuous amperometric or coulometric glucose sensor may located no more than a pre-determined distance away from the distal end. An insulin pump may be fluidically coupled to the proximal end, and may be attached to the insulin delivery cannula directly or via an intervening tube. The glucose sensor and the insulin delivery cannula may be configured to be inserted into the subcutaneous space simultaneously by a single insertion device.

### CROSS-REFERENCE

This application claims the benefit of U.S. Provisional Patent Application No. 63/181,083, filed Apr. 28, 2021, which is incorporated by reference herein in its entirety.

### BACKGROUND

Many persons with Type 1 or insulin-treated Type 2 diabetes may wish to perform continuous glucose monitoring (CGM) during insulin pump usage (continuous subcutaneous insulin infusion, CSII) wherein the insulin is delivered to the subcutaneous interstitial space via a cannula that resides in the subcutaneous interstitial space. The sensing portion of CGM devices may also reside in this subcutaneous interstitial space. Using currently-available technology, the through-the-skin insertion sites for these two devices (insulin cannula and CGM sensor) may need to be widely separated to avoid CGM interference from the insulin formulation.

### SUMMARY

The present disclosure provides methods and systems that may use concentrated insulin in order to decrease or eliminate glucose sensing artifact from CGM sensors that are deployed in the immediate vicinity of insulin delivery.

In an aspect, the present disclosure provides A system for managing blood glucose in a subject, comprising: an insulin delivery cannula comprising a hollow tube comprising a proximal end and a distal end, wherein the proximal end is in fluid communication with a source of a concentrated insulin or insulin analog formulation, wherein the concentrated insulin or insulin analog formulation has a concentration of at least about 150 units per milliliter (units/mL), wherein the distal end is configured to deliver the concentrated insulin or insulin analog formulation into a subcutaneous space of the subject; a glucose sensor located no more than a pre-determined distance away from the distal end, wherein the glucose sensor is a continuous amperometric or coulometric glucose sensor comprising at least one indicating electrode; and an insulin pump fluidically coupled to the proximal end of the insulin delivery cannula, wherein the insulin pump is attached to the insulin delivery cannula either directly or via an intervening tube, wherein the glucose sensor and the insulin delivery cannula are configured to be inserted into the subcutaneous space of the subject simultaneously by a single insertion device.

In some embodiments, the concentration is between 200 and 400 units/mL. In some embodiments, the concentration is at least 400 units/mL. In some embodiments, the concentration is 500 units/mL.

In some embodiments, the concentrated insulin or insulin analog formulation comprises an excipient comprising a phenolic compound or cresol. In some embodiments, the excipient comprises the phenolic compound. In some embodiments, the excipient comprises the cresol.

In some embodiments, the system further comprises a housing comprising an upper accessible surface and a lower surface configured to be adhered to a skin surface of the subject.

In some embodiments, the at least one indicating electrode comprises gold, carbon, graphite, platinum, or iridium.

In some embodiments, the pre-determined distance is about 15 millimeters (mm), about 14 mm, about 13 mm, about 12 mm, about 10 mm, about 9 mm, about 8 mm, about 7 mm, about 6 mm, about 5 mm, about 4.5 mm, about 4 mm, about 3.5 mm, about 3 mm, about 2.5 mm, about 2 mm, about 1.5 mm, or about 1 mm. In some embodiments, the pre-determined distance is about 7 mm. In some embodiments, the pre-determined distance is about 5 mm. In some embodiments, the pre-determined distance is about 2.5 mm.

In some embodiments, the glucose sensor comprises an electrode layer comprising the at least one indicating electrode, wherein the electrode layer underlies a redox-catalytic layer comprising (1) a redox mediator comprising a metal compound covalently bound to a ligand, and (2) an enzyme comprising glucose oxidase or glucose dehydrogenase. In some embodiments, the ligand is pyridine-based. In some embodiments, the ligand is 4,4′-dimethyl-2,2′-bipyridine. In some embodiments, the ligand is imidazole-based. In some embodiments, the redox mediator is bound to poly (4-vinyl pyridine). In some embodiments, the redox mediator is bound to poly (1-vinyl imidazole).

In some embodiments, the glucose sensor further comprises an insulating layer and a metal layer, wherein the insulating layer is coupled to the metal layer, and wherein the metal layer is coupled to the electrode layer. In some embodiments, the insulating layer comprises a polyimide or liquid crystal polymer. In some embodiments, the metal layer has a thickness of at least about 1 micrometer (μm), 2 μm, 3 μm, 4 μm, 5 μm, 6 μm, 7 μm, 8 μm, 9 μm, or 10 μm. In some embodiments, the metal layer comprises titanium, gold, or platinum.

In some embodiments, the electrode layer comprises a film having a thickness of no more than about 1000 nanometers (nm), 900 nm, 800 nm, 700 nm, 600 nm, 500 nm, 400 nm, 300 nm, 200 nm, or 100 nm.

In some embodiments, the redox mediator and the enzyme allow electron transfer from subcutaneous glucose to the at least one indicating electrode sufficient to cause a response of the glucose sensor to a subcutaneous glucose concentration of the subject at an applied bias potential of no more than about +300 millivolts (mV), +250 mV, +200 mV, +150 mV, +100 mV, or +50 mV relative to a reference electrode. In some embodiments, the applied bias potential of no more than about +250 mV, +200 mV, +150 mV, +100 mV, or +50 mV relative to the reference electrode allows the electrode layer to undergo substantially no electropolymerization of the excipient during continuous operation of at least one hour of the amperometric glucose sensor, thereby maintaining a sensitivity of the amperometric glucose sensor to the subcutaneous glucose concentration in presence of the concentrated insulin or insulin analog formulation.

In some embodiments, the metal compound comprises a metal selected from the group consisting of: Osmium, Ruthenium, Palladium, Platinum, Rhodium, Iridium, Cobalt, Iron, and Copper. In some embodiments, the metal compound comprises a metal selected from the group consisting of: Ruthenium, Palladium, Platinum, Rhodium, Iridium, Cobalt, Iron, and Copper.

In some embodiments, the ligand comprises a heterocyclic nitrogen compound, a pyridine ring combined with an imidazole ring, a non-nitrogen element substituted into a heterocycle ring, or an accessory “R” group bound to a heterocyclic ring. In some embodiments, the heterocyclic nitrogen compound comprises pyridine or imidazole with 1 ring, 2 rings, 3 rings, or 4 rings. In some embodiments, the reference electrode is a combined reference/counter electrode. In some embodiments, the reference electrode comprises a silver/silver chloride (Ag/AgCl) reference electrode.

The system of claim 1, wherein the glucose sensor is disposed on an outer wall of the hollow tube. The system of claim 1, wherein the glucose sensor is the continuous amperometric glucose sensor. The system of claim 1, wherein the glucose sensor is the continuous coulometric glucose sensor. The system of claim 1, wherein the insulin pump is a tube pump. The system of claim 1, wherein the insulin pump is a patch pump. The system of claim 1, further comprising a cartridge comprising the source of the concentrated insulin or insulin analog formulation.

In another aspect, the present disclosure provides a method for managing blood glucose in a subject, comprising: (a) obtaining a device for delivery of the insulin or insulin analog formulation and measurement of subcutaneous glucose concentration, wherein the device comprises: (i) a hollow tube comprising a proximal end and a distal end, wherein the proximal end is in fluid communication with a source of the concentrated insulin or insulin analog formulation, wherein the concentrated insulin or insulin analog formulation has a concentration of at least about 150 units per milliliter (units/mL), wherein the distal end is configured to deliver the concentrated insulin or insulin analog formulation into a subcutaneous space of the subject; and (ii) a glucose sensor located no more than a pre-determined distance away from the distal end, wherein the glucose sensor is a continuous amperometric or coulometric glucose sensor comprising at least one indicating electrode; (b) connecting the proximal end of the hollow tube to the source of the concentrated insulin or insulin analog formulation; (c) inserting the glucose sensor and the distal end of the hollow tube of the insulin delivery cannula into the subcutaneous space of the subject simultaneously by a single insertion device; and (d) simultaneously (1) delivering the concentrated insulin or insulin analog formulation into a subcutaneous space of the subject using an insulin pump fluidically coupled to the proximal end of the insulin delivery cannula, wherein the insulin pump is attached to the insulin delivery cannula either directly or via an intervening tube, and (2) measuring the subcutaneous glucose concentration of the subject.

In some embodiments, the concentration is between 200 and 400 units/mL. In some embodiments, the concentration is at least 400 units/mL. In some embodiments, the concentration is 500 units/mL.

In some embodiments, the concentrated insulin or insulin analog formulation comprises an excipient comprising a phenolic compound or cresol. In some embodiments, the excipient comprises the phenolic compound. In some embodiments, the excipient comprises the cresol.

In some embodiments, at least the device is enclosed within a housing comprising an upper accessible surface and a lower surface configured to be adhered to a skin surface of the subject.

In some embodiments, the at least one indicating electrode comprises gold, carbon, graphite, platinum, or iridium.

In some embodiments, the pre-determined distance is about 15 millimeters (mm), about 14 mm, about 13 mm, about 12 mm, about 10 mm, about 9 mm, about 8 mm, about 7 mm, about 6 mm, about 5 mm, about 4.5 mm, about 4 mm, about 3.5 mm, about 3 mm, about 2.5 mm, about 2 mm, about 1.5 mm, or about 1 mm. In some embodiments, the pre-determined distance is about 7 mm. In some embodiments, the pre-determined distance is about 5 mm. In some embodiments, the pre-determined distance is about 2.5 mm.

In some embodiments, the glucose sensor comprises an electrode layer comprising the at least one indicating electrode, wherein the electrode layer underlies a redox-catalytic layer comprising (1) a redox mediator comprising a metal compound covalently bound to a ligand, and (2) an enzyme comprising glucose oxidase or glucose dehydrogenase. In some embodiments, the ligand is pyridine-based. In some embodiments, the ligand is 4,4′-dimethyl-2,2′-bipyridine. In some embodiments, the ligand is imidazole-based. In some embodiments, the redox mediator is bound to poly (4-vinyl pyridine). In some embodiments, the redox mediator is bound to poly (1-vinyl imidazole).

In some embodiments, the glucose sensor further comprises an insulating layer and a metal layer, wherein the insulating layer is coupled to the metal layer, and wherein the metal layer is coupled to the electrode layer. In some embodiments, the insulating layer comprises a polyimide or liquid crystal polymer. In some embodiments, the metal layer has a thickness of at least about 1 micrometer (μm), 2 μm, 3 μm, 4 μm, 5 μm, 6 μm, 7 μm, 8 μm, 9 μm, or 10 μm. In some embodiments, the metal layer comprises titanium, gold, or platinum.

In some embodiments, the electrode layer comprises a film having a thickness of no more than about 1000 nanometers (nm), 900 nm, 800 nm, 700 nm, 600 nm, 500 nm, 400 nm, 300 nm, 200 nm, or 100 nm.

In some embodiments, the redox mediator and the enzyme allow electron transfer from subcutaneous glucose to the at least one indicating electrode sufficient to cause a response of the glucose sensor to a subcutaneous glucose concentration of the subject at an applied bias potential of no more than about +300 millivolts (mV), +250 mV, +200 mV, +150 mV, +100 mV, or +50 mV relative to a reference electrode. In some embodiments, the applied bias potential of no more than about +250 mV, +200 mV, +150 mV, +100 mV, or +50 mV relative to the reference electrode allows the electrode layer to undergo substantially no electropolymerization of the excipient during continuous operation of at least one hour of the amperometric glucose sensor, thereby maintaining a sensitivity of the amperometric glucose sensor to the subcutaneous glucose concentration in presence of the concentrated insulin or insulin analog formulation.

In some embodiments, the metal compound comprises a metal selected from the group consisting of: Osmium, Ruthenium, Palladium, Platinum, Rhodium, Iridium, Cobalt, Iron, and Copper. In some embodiments, the metal compound comprises a metal selected from the group consisting of: Ruthenium, Palladium, Platinum, Rhodium, Iridium, Cobalt, Iron, and Copper.

In some embodiments, the ligand comprises a heterocyclic nitrogen compound, a pyridine ring combined with an imidazole ring, a non-nitrogen element substituted into a heterocycle ring, or an accessory “R” group bound to a heterocyclic ring. In some embodiments, the heterocyclic nitrogen compound comprises pyridine or imidazole with 1 ring, 2 rings, 3 rings, or 4 rings. In some embodiments, the reference electrode is a combined reference/counter electrode. In some embodiments, the reference electrode comprises a silver/silver chloride (Ag/AgCl) reference electrode.

In some embodiments, the glucose sensor is disposed on an outer wall of the hollow tube. In some embodiments, the glucose sensor is the continuous amperometric glucose sensor. In some embodiments, the glucose sensor is the continuous coulometric glucose sensor. In some embodiments, the insulin pump is a tube pump. In some embodiments, the insulin pump is a patch pump. In some embodiments, the method further comprises establishing fluid communication between the proximal end and a cartridge comprising the source of the concentrated insulin or insulin analog formulation.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “Figure” and “FIG.” herein), of which:

Embodiments will be readily understood by the following detailed description in conjunction with the accompanying drawings and the appended claims. Embodiments are illustrated by way of example and not by way of limitation in the figures of the accompanying drawings.

FIG. 1 illustrates an example of a benefit of using concentrated insulin in high-bias sensors such as those that estimate glucose concentration by measurement of the oxidation of hydrogen peroxide. The left panel shows the location of a glucose sensor (checkerboard triangle) in terms of how far it must be separated from an insulin delivery cannula (white triangle) to avoid measurement errors caused by the insulin bolus. During a meal bolus of standard U100 (100 units/milliliter (mL)) insulin, the glucose sensor may need to be located at a substantial distance from the insulin delivery cannula to avoid the adverse effects of the insulin formulation (e.g., spurious glucose measurement signal). These adverse effects may be caused by the presence of phenolic preservatives which may be commonly present in insulin formulations. The gray disk shows the subcutaneous distance over which the insulin diffuses before its absorption into the bloodstream, e.g., the “region of insulin influence”. The right panel shows the situation in which concentrated insulin (for example, 500 units/mL insulin) is used instead. Since there is a much lower radius of insulin distribution, the sensor can be located much closer to the insulin delivery site.

FIG. 2 is a line graph illustrating an example of glucose measurement data obtained from a person with Type 1 diabetes in whom an redox-mediated glucose-sensing cannula (with a low polarizing bias voltage) is present in the subcutaneous space. Such a device has both glucose-sensing and insulin delivery functionalities. The solid triangle symbols show reference blood glucose values, as measured every 5 minutes by a reference instrument, the YSI 2300 glucose analyzer. The solid line shows continuous data from this low bias glucose sensor located on the cannula (several millimeters proximal to the distal insulin-delivering tip). At time=15 minutes, a bolus of as part insulin (100 units/mL) is delivered to the person subcutaneously through the cannula. An error (artifact) is observed, which is caused by dilution of glucose in the interstitial space, immediately after this meal bolus. The error is characterized by downward travel of the sensed glucose signal. This artifact is resolved, and the signal returns back to baseline, about 10-15 minutes after delivery of the meal bolus.

FIG. 3 illustrates an example of a low bias redox-mediated glucose sensor integrated into an insulin delivery cannula (black device) in the subcutaneous space. In the upper panel, the gray disk shows the region of influence of 100 unit/mL insulin (approx. 10 units) resulting from the outward dispersal of the insulin bolus in the subcutaneous space. The time at which the bolus is given is indicated by the black arrow on the line graph. Because the volume of this bolus is large, the insulin formulation remains in contact with the sensor for some time before it is absorbed; thus, the magnitude of the dilution artifact is large and its duration is relatively long, as depicted in the line graph. The lower panel shows the contrasting situation when the same insulin dose (same number of units) is given but the formulation is concentrated, in this example: 500 units/mL insulin. In this case, because the liquid volume is small, the magnitude and duration of the downward dilution errors are small, as depicted in the line graph on the right.

FIG. 4 is a graph illustrating an example of how changing the volume of insulin delivery (as modeled by a tissue distribution in the shape of a disk) changes the radius of said disk in the case of a bolus of 15 units of insulin. Assume that a CGM sensor is placed adjacent to (not above or below in a vertical axis) the insulin delivery site. In order to avoid the dilution artifact, the CGM must be placed at a point beyond the radius of this disk. As one examines the decline in the radius curve with increasing concentration of insulin, the graph shows that increasing concentration has a very large beneficial effect in reducing the disk radius. For example, the radius of insulin distribution is about 7 mm for 100 units/mL insulin concentration, but only about 3 mm for 500 units/mL insulin concentration. For data on this graph, the height of the insulin distribution disk was assumed to be fixed at 1 mm.

FIG. 5 is a graph illustrating an example of how different bolus sizes affect the radius of the disk of insulin distribution, assuming that the height of the disk is fixed at 1 mm. The graph shows that two major variables affect the disk radius: the concentration of insulin (e.g., as shown in FIG. 4), and also the size of the bolus. For 500 units/mL insulin concentration, for example, a bolus of 7.5 units yields a disk radius of slightly over 2 mm, as compared to a radius of about 4.5 mm for a bolus of 30 units. From this graph, one may appreciate that concentrated insulin has more valuable contribution when the boluses are large. In the example for a 30 unit bolus, separate CGM may need to be located almost 10 mm away from the site of insulin delivery in the horizontal plane (parallel to the skin surface) in the case of 100 units/mL insulin concentration, but only 4.5 mm away from the site in the case of 500 units/mL insulin concentration. In particular, one finding deserves special mention: for a concentration of 200 units/mL (or higher), a distance of 7 mm is sufficient to avoid sensing error from dilution, even for bolus doses as high as 30 units.

FIG. 6 illustrates an example of an embodiment of a combination sensor and cannula. On the left is shown the indicating electrode surface of the sensing cannula, which is approximately 14.5 mm in total length, excluding the proximal extension of the fluid path tube, which can be seen to extend proximally beyond the limit of the indicating electrode. Note also that there is an inner tube that extends through the length of the device, allowing an insulin formulation to be delivered subcutaneously at the distal end. The location of the line which divides the above-skin part from the below-skin part is given by the dashed line. Typically, the skin surface is about 7 mm from the distal end of the device. In some embodiments, the surface of the indicating electrode is gold. On the right is shown the opposite surface, which is the reference electrode surface (sometimes, in a two electrode configuration such as this, which may be referred to as a combined reference-counter electrode). In some embodiments, this surface is a silver (Ag) layer chloridized with silver chloride (AgCl).

### DETAILED DESCRIPTION

References are made herein to the accompanying drawings which form a part hereof, and in which are shown by way of illustration embodiments that may be practiced. It is to be understood that other embodiments may be utilized and structural or logical changes may be made without departing from the scope. Therefore, the following detailed description is not to be taken in a limiting sense.

Various operations may be described as multiple discrete operations in turn, in a manner that may be helpful in understanding embodiments; however, the order of description should not be construed to imply that these operations are order dependent.

The description may use perspective-based descriptions such as up/down, back/front, and top/bottom. Such descriptions are merely used to facilitate the discussion and are not intended to restrict the application of disclosed embodiments.

As used herein, the term “cannula” or “insulin delivery cannula” generally refers to a hollow tube (e.g., round, flattened, or other in cross section) that terminates in the subcutaneous space of a human and is intended to serve as a conduit through which insulin or insulin analog can be infused or injected. The cannula may be fabricated using a rigid material, such as a polymer or a metal, having an interior (e.g., inner) surface and an exterior (e.g., outer) surface, and an opening at both ends.

As used herein, the term “sensing cannula” generally refers to a cannula having an analyte sensor disposed on the exterior surface and one or more fluid delivery channels contained within the cannula.

As used herein, the term “concentrated insulin” or “concentrated insulin formulation” generally refers to a formulation of insulin or insulin analog whose concentration is greater than the standard 100 units/mL (U100). For example, concentrated insulin may have a concentration of 150 units/mL, 200 units/mL, 250 units/mL, 300 units/mL, 350 units/mL, 400 units/mL, 450 units/mL, 500 units/mL, or greater than 500 units/mL.

As used herein, the term “continuous glucose monitor (CGM)” generally refers to a system comprising electronics configured for continuous or nearly continuous measurement of glucose levels from a subject (e.g., a human being, an animal, or a mammal) and/or reporting of such measurements.

As used herein, the term “CGM injection port” generally refers to a device (e.g., a unified device) configured for use on the skin of a subject (e.g., a human being, an animal, or a mammal) having a combination of a sensor and a cannula that includes an electrical interface to signal acquisition electronics and a port for attachment of a fluid source such as an insulin pen, a syringe, or another fluid delivery device.

As used herein, the term “CGM infusion set” generally refers to a device (e.g., a unified device) configured for use on the skin of a subject (e.g., a human being, an animal, or a mammal) having a combination of a sensor and a cannula that includes an electrical interface to signal acquisition electronics and a port for attachment of a fluid source such as a pump or a gravity-fed sourced source.

The terms “coupled” and “connected,” along with their derivatives, may be used herein. It should be understood that these terms are not intended as synonyms for each other. Rather, in particular embodiments, “connected” may be used to indicate that two or more elements are in direct physical or electrical contact with each other. “Coupled” may be used to indicate that two or more elements are in direct physical or electrical contact. However, “coupled” may also be used to indicate that two or more elements are not in direct contact with each other, but yet still cooperate or interact with each other.

As used herein, a phrase in the form “A/B” or in the form “A and/or B” means (A), (B), or (A and B). For the purposes of the description, a phrase in the form “at least one of A, B, and C” means (A), (B), (C), (A and B), (A and C), (B and C), or (A, B, and C). For the purposes of the description, a phrase in the form “(A)B” means (B) or (AB) that is, A is an optional element.

As used herein, the terms “embodiment” or “embodiments,” may each refer to one or more of the same or different embodiments. Furthermore, the terms “comprising,” “including,” “having,” and the like, as used with respect to embodiments, are synonymous, and are generally intended as “open” terms (e.g., the term “including” should be interpreted as “including but not limited to,” the term “having” should be interpreted as “having at least,” the term “includes” should be interpreted as “includes but is not limited to,” etc.).

With respect to the use of any plural and/or singular terms herein, the plural can be translated to the singular and/or the singular can be translated to the plural, as is appropriate to the context and/or application. The various singular/plural permutations may be expressly set forth herein for sake of clarity.

Persons with Type 1 and Type 2 diabetes (e.g., diabetes patients) may be at risk for complications that are related to poor control of their blood glucose levels. They may be susceptible to various complications such as eye disease and/or blindness, kidney disease, nerve disease, limb amputations, atherosclerotic heart and vascular disease, etc. For this reason, many such persons (e.g., diabetes patients) attempt to keep their glucose levels as close to normal as possible in hopes of avoiding these complications. Many persons with Type 1 diabetes (and an increasing number of those with Type 2 diabetes) deliver insulin by the use of an insulin pump, a process which may be referred to as continuous subcutaneous insulin infusion (CSII). This continuous delivery mimics the continuous delivery of insulin in a non-diabetic person, which is carried out by the beta cells of the endocrine pancreas. Using CSII to safely manage insulin dosing may be accompanied by knowledge of one's glucose level. The use of CSII without such knowledge may be dangerous. Although some diabetes patients are able to maintain satisfactory control of their blood glucose with the use of blood glucose monitoring fingersticks and strips, a better method of monitoring glucose is by the use of continuous glucose monitoring (CGM).

Most insulin formulations sold worldwide may have a concentration of 100 units per mL, and this concentration is also known as U100. However, some companies have developed more concentrated insulins, as described by, for example, (Schloot, Hood, Corrigan et al Concentrated insulins in current clinical practice. Diabetes Research and Clinical Practice 148(2019) 93-101.), which is incorporated by reference herein in its entirety. For example, Lilly, Inc. has developed a 200 unit/mL lis-pro insulin, and this insulin is stable for 28 days at room temperature. It is bio-equivalent to 100 unit/mL in terms of its activity. For many years, Lilly has also marketed a 500 unit/mL highly concentrated R insulin. Other companies that provide concentrated insulin formulations include Novo, Sanofi, Thermalin, and others.

Chemical Interference Vs Dilution Interference

When the cannula and CGM sensor are placed close to one another in a mammalian body, there may be two types of interference that can occur. One type is chemical interference in the CGM process. This interference may arise from the presence of phenolic-based preservatives in all insulin formulations, as described by, for example, (Ward W K, Heinrich G et al: An Amperometric Glucose Sensor Integrated into an Insulin Delivery Cannula: In Vitro and In Vivo Evaluation. Diabetes Technology & Therapeutics. April 2017.226-236), which is incorporated by reference herein in its entirety. This type of interference may be caused by oxidation of phenol and/or metal-cresol (initially causing a spurious glucose measurement signal that simulates hyperglycemia, and eventually resulting in permanent decay of the electrochemical signal) and can be avoided by the use of a low polarizing bias redox-mediated glucose sensing system, which is also described by, for example, U.S. Pat. No. 10,780,222 (Ward et al), which is incorporated by reference herein in its entirety. These sensors can be referred to as low bias sensors. These sensors may comprise redox mediators that acquire electrons from glucose and donate those electrons to an indicating electrode. Other CGM sensors may be designed so that glucose and oxygen combine to form hydrogen peroxide, the concentration of which is proportional to the tissue glucose concentration. These sensors may be operated at a relatively high bias in order to pull the electrons from the peroxide; therefore these sensors may be referred to as high bias sensors.

The second type of interference is much different and may be referred to as dilution interference. This interference may be caused by placing a volume of liquid into the subcutaneous interstitial space, thus diluting the glucose and reducing its concentration. This interference may pose an issue for both low bias sensors and high bias sensors. Concentration is defined as the amount of solute per amount of solute plus solvent. Dilution interference may result from the addition of more water-based solvent without changing the amount of solute (glucose); such an effect results in the artifact of an inappropriately reduced glucose concentration.

The present disclosure provides devices, systems, and methods that concentrated insulin formulations; such formulations may effectively address both of these two causes of interference in CGM: chemical interference and dilution interference.

Devices, systems, and methods of the present disclosure may be suited for use with the specific concentration of phenolic preservatives in commercially-available insulin preparations. The different marketed insulin preparations may contain 2-3.5 milligrams per milliliter (mg/mL) of total phenolics (phenol and meta-cresol). As a specific example, for Humulin R insulin 100 units/mL (from Lilly Corp), there are 2.5 mg/mL of total phenolics. Notably, for Lilly's concentrated Humulin R insulin (500 units/mL), there is also 2.5 mg/mL of total phenolics. This is a key point. Despite the insulin having 5 times as much insulin per mL, the concentration of phenolics found to be sufficient was the same as for the standard 100 units/mL insulin.

Mechanism of Benefit in High Bias Sensors

This disparity in the ratio of preservatives to insulin is the basis for some embodiments of the present disclosure. Because the chemical interference is based on the concentration of phenolics, there is less chemical interference when concentrated insulin is given, whether or not the sensor is a high bias or a low bias sensor. However, since a low bias sensor does not register interference from the phenolics, this embodiment is especially suited for use in high bias sensors. Stated differently, the use of concentrated insulin may effectively reduce the amount of preservative in each bolus dose of insulin. The larger bolus doses of insulin are more likely to cause interference than the slow, low rate basal delivery of insulin.

This benefit may arise due to the fact that when insulin is concentrated, such as in Lilly 500 unit/mL Regular insulin, the concentration of the phenolic preservatives is NOT increased commensurate with the increased insulin concentration. In other words, the phenolic concentration is related to the volume, not to the concentration, of insulin.

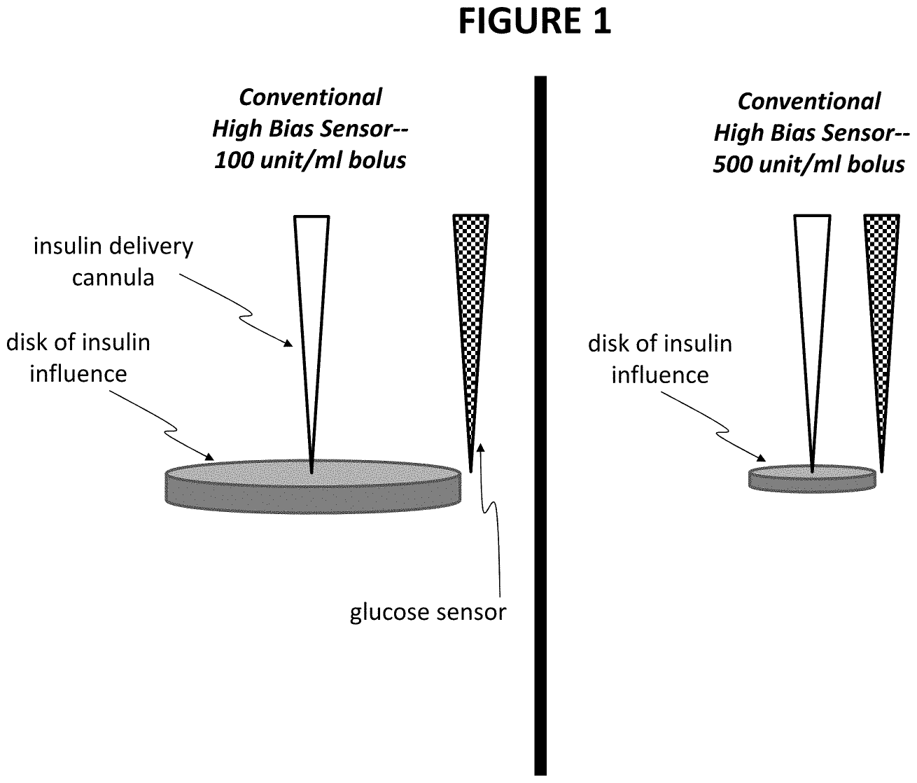

One benefit of using concentrated insulin in high bias sensors arises from reducing the amount of preservatives in concept. FIG. 1 illustrates an example of a benefit of using concentrated insulin in high-bias sensors such as those that estimate glucose concentration by measurement of the oxidation of hydrogen peroxide. The left panel of FIG. 1 shows the location of a glucose sensor (a low bias sensor that measures the generation of hydrogen peroxide; checkerboard triangle) in terms of how far it must be separated from an insulin delivery cannula (white triangle) to avoid measurement errors caused by the insulin bolus. During a generous meal bolus of standard U100 (100 units/mL) insulin, the glucose sensor may need to be located at a substantial distance (e.g., greater than 7 mm away) from the insulin delivery cannula to avoid the adverse effects of the insulin formulation (e.g., spurious glucose measurement signal). These adverse effects may be caused by the presence of phenolic preservatives which may be commonly present in insulin formulations. This distance is described by, for example, (O'Neal et al, Feasibility of Adjacent Insulin Infusion and Continuous Glucose Monitoring via the Medtronic Combo-Set. J Diabetes Sci Technol 2013; 7(2):381-388), which is incorporated by reference herein in its entirety. A peroxide-measuring glucose sensor may need to be separated by at least 7 mm from a cannula that delivers standard 100 unit/mL insulin in the volume needed for a standard meal bolus. More specifically, when a bolus of typical 100 unit/mL insulin is given, it diffuses out into the subcutaneous interstitial space and remains there until it is absorbed into the bloodstream. The gray disk shows the subcutaneous distance over which the insulin diffuses before its absorption into the bloodstream, e.g., the “region of insulin influence”. Since the preservatives in the insulin may cause significant errors in the accuracy of continuous glucose measurement, the sensor must be placed outside of this region of “insulin influence.”

The right panel of FIG. 1 illustrates an example of the benefit of using concentrated insulin when one uses a conventional glucose sensor that measures the generation of peroxide. When a comparable dose of concentrated insulin (for example, 500 unit/mL insulin) is given instead, there is a much lower concentration of phenolic insulin preservative in the region of insulin influence. Therefore, the sensor can be located much closer to the insulin delivery cannula. This close location is more convenient for the user, since the patient can use a single polymer base adhesive and a single covering bandage for the two devices. In addition, the sensor and the insulin delivery cannula consequently can be conveniently located on a single platform and inserted concurrently. By being located on the same platform, a single insertion device can be used for both devices.

Peroxide-measuring sensors include those manufactured or sold by Dexcom, Medtronic, Agamatrix (Waveform), and others. The indicating (working) electrode(s) in peroxide-measuring sensors can be made of platinum or certain other elements or compounds. There can be a counter electrode and a reference electrode or these types of electrodes can be combined, sometimes referred to as a pseudo-reference electrode. The enzyme (e.g., glucose oxidase or glucose dehydrogenase) can be deposited directly on the indicating electrode, or there can be a layer between the indicating electrode and the enzyme. In some cases, this intervening layer can ensure that acetaminophen, ascorbic acid, and other interfering compounds are kept away from the indicating electrode, where, if they are oxidized, they can cause a spurious or abnormal glucose-like signal. Covering the enzyme layer is a membrane that allows passage of both oxygen and glucose. This membrane, sometimes referred to as an outer membrane, can be made of polyurethane or other polymer. In some embodiments, this membrane is more permeable to oxygen than to glucose, since oxygen may be needed as a substrate for the production of hydrogen peroxide.

Phenolics in Insulin Preparations

Given the adverse role that phenolic preservatives have in causing glucose-like currents and electrode deactivation (with signal decay), pharmaceutical scientists may be motivated to simply find another preservative, one that is not oxidized at bias potentials used in CGM sensors. Such an exclusion, however, may pose problems for at least the following reason: the phenolic compounds have an important role separate from the role as anti-microbial preservatives, namely that of biochemical stabilization. The insulin protein does not exist in pharmaceutical preparations as monomeric insulin. Instead, it exists as hexameric insulin, that is, a spherical oligomer. This hexameric configuration is important because it preserves the biochemical action of insulin and avoids chemical degradation (for example, degradation to a fibrillated, misfolded, aggregated protein). The phenolic compounds in insulin may effectively prevent such degradation. In other words, if one removes the phenolics from insulin, a risk for microbial contamination may be introduced; moreover, loss of the stabilizing hexameric configuration may occur.

Use of Concentrated Insulin in Low Bias Glucose Sensors

Low bias insulin sensors may avoid spurious measurement signals arising from the interference from phenolics because they avoid oxidation of the phenolics. By combining the sensor and the cannula, the inconvenience and discomfort of using two percutaneous devices may be avoided. The integration of a CGM and an insulin delivery cannula into a single device is described by, for example, (Ward W K, Heinrich G et al: An Amperometric Glucose Sensor Integrated into an Insulin Delivery Cannula: In Vitro and In Vivo Evaluation, Diabetes Technology & Therapeutics. April 2017.226-236), which is incorporated by reference herein in its entirety. This integrated device may comprise a mediator-based glucose sensor that has been embedded in, or placed on, the outer wall of an insulin cannula. By depositing a redox mediator (such as osmium, ruthenium or others), a pyridine- or imidazole-based ligand, and glucose oxidase on the sensor's indicating electrode, and applying a low potential bias (e.g., less than 175 mV), this CGM sensor can function in the direct presence of insulin and insulin analogs despite the presence of phenolic-type preservatives in the insulin formulation. These preservatives may cause issues arising from marked inaccuracy in conventional peroxide-sensing high bias sensors when glucose sensing is attempted in the direct presence of an insulin formulation.

Although the use of a low-bias sensor may avoid artifact caused by the phenolics, it does not avoid artifact caused by the dilution effect. For example, a study in humans demonstrated glucose sensing accuracy during the use of a unified CGM/insulin delivery cannula, as described by, for example, (Jacobs P G, Tyler N S, Vanderwerf S M, et al. Measuring glucose at the site of insulin delivery with a redox-mediated sensor. Biosens Bioelectron. 2020 Oct. 1; 165), which is incorporated by reference herein in its entirety. Confirming the benefit of the low bias, there was no difference between the magnitude and duration of the artifact (error) caused by insulin delivery vs delivery of the same volume of saline. However, there was an error that was present both in the insulin and in the saline control groups: a dilution error caused by dilution of the subcutaneous interstitial space with a water-based liquid. This error was due to temporarily diluting the interstitial glucose concentration—the error in glucose sensor measurement persisted for 5-20 minutes after the delivery of the insulin bolus, until the subcutaneous tissue could fully absorb the excess fluid.

These results demonstrated that there was an artifact (error) in both insulin and saline control groups; the error was not eliminated by use of a low bias sensor. The magnitude of the error in the insulin and saline groups were identical. In other words, there was no physiologic effect of the hormone and no specific error caused by excipients such as preservatives in its formulation. The only error in both cases was one of dilution. This dilution artifact was usually in the negative direction, indicating transient diluting of the interstitial glucose to a lower concentration. Further analysis of the results indicates that the only pertinent variable that affects the magnitude of the error is the volume of the liquid that was administered. The error from such dilution was directly proportional to the volume of the liquid that is administered during a bolus (e.g., boluses given for meals or snacks, correction boluses given to reduce hyperglycemia, or basal microboluses).

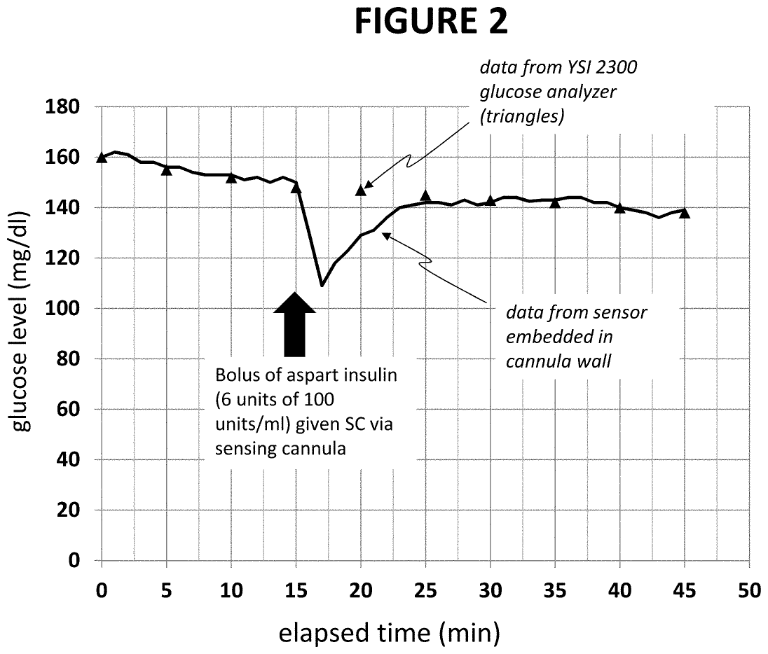

FIG. 2 is a line graph illustrating an example of glucose measurement data obtained from a person with Type 1 diabetes in whom an redox-mediated glucose-sensing cannula (with a low polarizing bias voltage) is present in the subcutaneous space. Such a device has both glucose-sensing and insulin delivery functionalities. The solid triangle symbols show reference blood glucose values, as measured every 5 minutes by a reference instrument, the YSI 2300 glucose analyzer. The solid line shows continuous data from this low bias glucose sensor located on the cannula (several millimeters proximal to the distal insulin-delivering tip). At time=15 minutes, a bolus of aspart insulin (100 units/mL) is delivered to the person subcutaneously through the cannula. An error (artifact) is observed, which is caused by dilution of glucose in the interstitial space, immediately after this meal bolus. The error is characterized by downward travel of the sensed glucose signal. This artifact is resolved, and the signal returns back to baseline, about 10-15 minutes after delivery of the meal bolus.

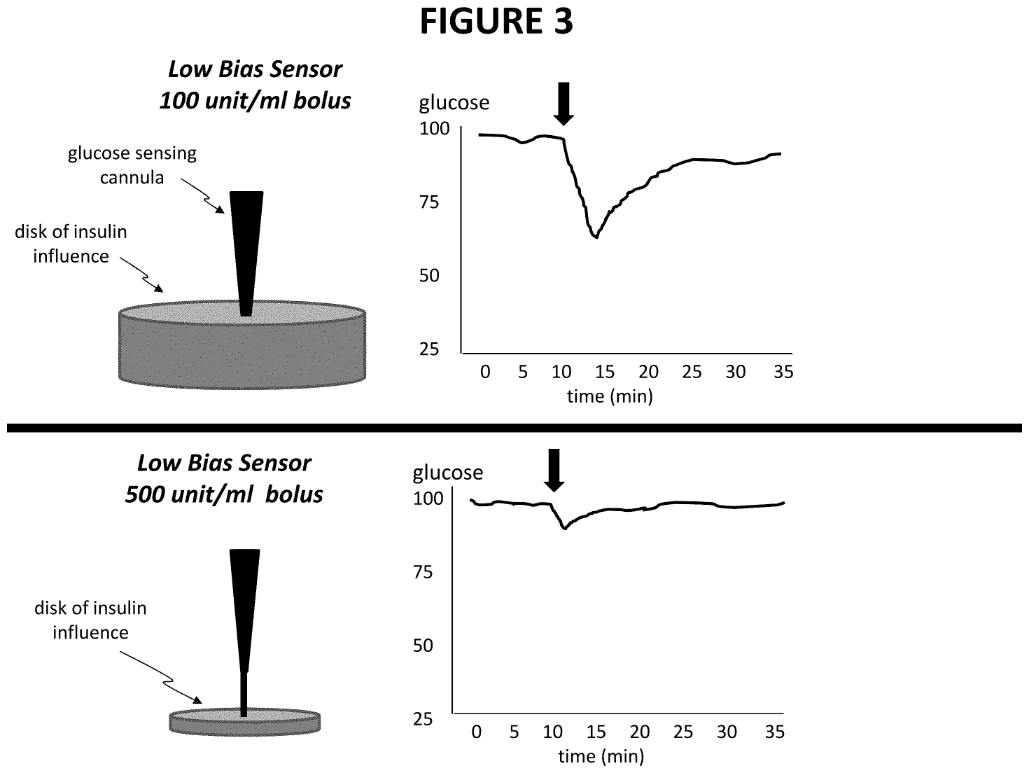

FIG. 3 illustrates an example of a low bias redox-mediated glucose sensor integrated into an insulin delivery cannula (black device) in the subcutaneous space. In the upper panel, the gray disk shows the region of influence of 100 unit/mL insulin (approx. 10 units) resulting from the outward dispersal of the insulin bolus in the subcutaneous space. The time at which the bolus is given is indicated by the black arrow on the line graph. Because the volume of this bolus is large, the insulin formulation remains in contact with the sensor for some time before it is absorbed; thus, the magnitude of the dilution artifact is large and its duration is relatively long, as depicted in the line graph. The lower panel shows the contrasting situation when the same insulin dose (same number of units) is given but the formulation is concentrated, in this example: 500 units/mL insulin. In this case, because the liquid volume is small, the magnitude and duration of the downward dilution errors are small, as depicted in the line graph on the right.

One simple way to measure the error from diluting the interstitial glucose concentration is to calculate the area under the curve (AUC), that is, the area under the baseline sensed glucose signal as a result of the dilution. There are two main differences between a small bolus and a large bolus. One, the signal during the error from a small bolus does not decline to as low a level as for a large bolus. Two, the signal for small bolus returns to the baseline more quickly than that of a large bolus. So, the difference between a large and small bolus may be evaluated by measuring how far the signal declines. Another approach is to measure the time that it takes to return to baseline (recovery time). However, the benefit of using AUC measurement is that it takes both metrics into account and allows for a more simple (single) metric that includes elements of the signal depth and time to recovery.

By determining the AUC in data from glucose-sensing cannulae, the dilutive error defined by AUC is observed to be largely proportional to the volume of the insulin formulation. That is, the error from delivering a 500 unit/mL formulation is approximately 80% less than the error of an equal dose of a 100 unit/mL formulation.

In some embodiments, a concentrated insulin formulation is loaded into the reservoir of an insulin pump and delivered to the diabetes patient via a combined insulin delivery cannula and glucose sensor (in which the sensor is embedded into, or placed on to the wall of the cannula). The concentrated insulin is delivered in both the basal and bolus states. Such use of concentrated insulin provides several benefits for the patient. First, the glucose measurement error is lower in magnitude and lasts for a shorter period of time compared to the use of standard non-concentrated insulin. Consequently, if the algorithm used to convert sensor current to a glucose measurement suspends provision of glucose information to the patient after a bolus in order to avoid spurious glucose signal readings, this suspension time can be quite short, thus avoiding dangerous prolonged periods of time during which the patient has no access to his or her continuous glucose data. Second, a patient can hold more units of concentrated insulin in his pump reservoir compared to standard non-concentrated insulin. Therefore, the duration of the time between the need to load fresh insulin into the reservoir can be lengthened (e.g., this is especially important to patients whose tissues are resistant to insulin and who require large insulin dosages). Third, the use of concentrated insulin allows for a smaller reservoir than for non-concentrated insulin. This can be important for people who wish to have a small form factor for their belt-worn insulin pump or their on-the-body patch pump.

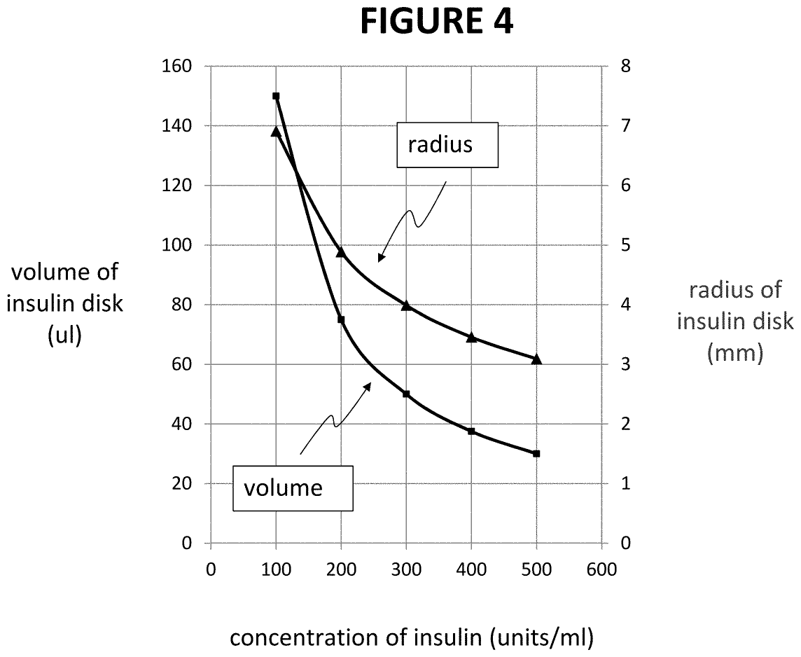

FIG. 4 is a graph illustrating an example of how changing the volume of insulin delivery (as modeled by a tissue distribution in the shape of a disk) changes the radius of said disk in the case of a bolus of 15 units of insulin. Assume that a CGM sensor is placed adjacent to (not above or below in a vertical axis) the insulin delivery site. In order to avoid the dilution artifact, the CGM must be placed at a point beyond the radius of this disk. As one examines the decline in the radius curve with increasing concentration of insulin, the graph shows that increasing concentration has a very large beneficial effect in reducing the disk radius. For example, the radius of insulin distribution is about 7 mm for 100 units/mL insulin concentration, but only about 3 mm for 500 units/mL insulin concentration. For data on this graph, the height of the insulin distribution disk was assumed to be fixed at 1 mm.

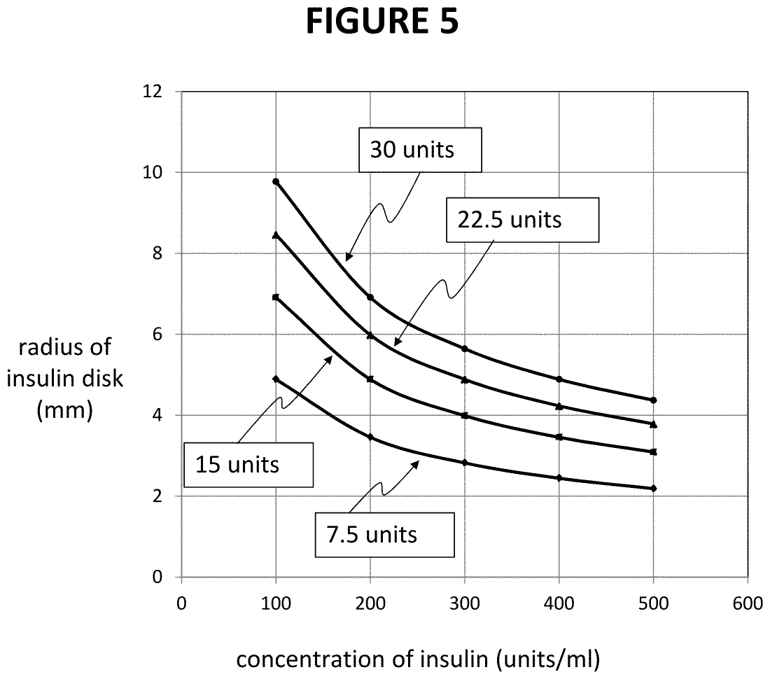

FIG. 5 is a graph illustrating an example of how different bolus sizes affect the radius of the disk of insulin distribution, assuming that the height of the disk is fixed at 1 mm. The graph shows that two major variables affect the disk radius: the concentration of insulin (e.g., as shown in FIG. 4), and also the size of the bolus. For 500 units/mL insulin concentration, for example, a bolus of 7.5 units yields a disk radius of slightly over 2 mm, as compared to a radius of about 4.5 mm for a bolus of 30 units. From this graph, one may appreciate that concentrated insulin has more valuable contribution when the boluses are large. In the example for a 30 unit bolus, separate CGM may need to be located almost 10 mm away from the site of insulin delivery in the horizontal plane (parallel to the skin surface) in the case of 100 units/mL insulin concentration, but only 4.5 mm away from the site in the case of 500 units/mL insulin concentration. In particular, one finding deserves special mention: for a concentration of 200 units/mL (or higher), a distance of 7 mm is sufficient to avoid sensing error from dilution, even for bolus doses as high as 30 units.

Fabrication of Glucose-Sensing Elements (e.g., in the Outer Wall of an Insulin Delivery Cannula)

In order to minimize the interference by reducing the bias potential, a redox-mediated chemistry scheme may be used. For example, the specific use of low bias, redox-based sensing technology to avoid preservative artifact is described in U.S. Pat. No. 10,780,222 (Ward et al), which is incorporated by reference herein in its entirety.

Osmium, ruthenium, and other compounds may be suitable for accepting electrons from glucose oxidase, more specifically from the prosthetic group of glucose oxidase known as flavin adenine dinucleotide (FAD). In one embodiment, the osmium is held in placed (coordinated) by its bond to a ligand such as, 4,4′-dimethyl 2,2′-bipyridine. Many other ligands can be used. Various electron rich chemical groups such as methyl, methoxy or amino, when bound to the pyridine or imidazole ligands, may allow the osmium to transfer electrons at a low polarizing bias. In some cases, the ligands, which coordinate, and bind to, the osmium redox centers, may be bound to a polymer, and this complex of mediator, ligand and polymer may be referred to as the redox mediator polymer (RMP).

In one embodiment, osmium is the redox mediator. The polymer backbone of the RMP comprises poly(l-vinyl imidazole) (PVI). Two coordination ligands, 4,4′-dimethyl,2,2′-bipyridine may be bound to an osmium group. The osmium may be bound to approximately one of every 5 to 15 imidazole groups on the PVI.

In one embodiment, the RMP is deposited on a gold indicating electrode, but other metals can be used, such as vitreous carbon, glassy carbon, graphite, platinum, or iridium. It is also possible to make the indicating electrode porous, for example by the use of acid anodization, laser poration, or plasma etching.

The enzyme can be glucose oxidase or glucose dehydrogenase, and is in contact with the osmium or other mediator, the ligand and the RMP. The enzyme can be extended by the use of albumin, collagen carrageenan, or other extending compound. In one embodiment, a gold indicating electrode is coated with glucose oxidase and bovine serum albumin (BSA) and crosslinked with glutaraldehyde or a suberimidate compound, then coated with an outer membrane. A requirement may be that glucose must be able to permeate through the outer membrane. In one embodiment, the RMP and the enzyme are coated with a polymeric outer membrane. Oxygen permeability may not be necessary for the function of this type of sensor, but a degree of glucose permeability may be necessary. The outer membrane can be made of polyurethane, Nafion, poly(vinylpyridine), poly(vinylpyridine)-co-styrene, molecular weight cutoff polymeric membranes, silicone, hydrogels and many other materials that allow glucose permeation.

For example, a gold indicating electrode, coated with RMP and crosslinked glucose oxidase and polarized at 0-180 mV vs a Ag/AgCl reference electrode, is able to measure glucose with little or no interference from the preservatives used in insulin formulations. In contrast, the use of a platinum sensor, coated with crosslinked glucose oxidase and polarized at 400-750 mV, undergoes a very large oxidation current when exposed to phenolics. Furthermore, if such exposure lasts for more than a few minutes, the electrode is consistently poisoned by a dense layer of electropolymerized phenolic compound that prevents H₂O₂ and other common analytes from reaching the indicating electrode and being measured. This process results in a slow decay of the sensor signal.

One method of creating a continuous sensor built into the wall of an insulin infusion cannula is to laminate flexible thin metal films on the outer wall of a hollow tubular structure that serves as the conduit for the insulin formulation. This conduit (cannula) it may be made from a polymer, but can also be a metal in some designs. The polymer can be polyurethane, polyethylene, silicone, or many other polymerized compounds. In some cases, the cannula is flexible, but also can be rigid.

In some cases, it may be desirable to place a metallic foil between the thin film metal electrodes and the polymer that makes up the insulin delivery cannula. In some construction designs, the foil can enhance durability and fatigue resistance, though the presence of the foil is not necessary. The use of the term “foil” indicates a metal layer that is at least 2 micrometers (μall) in thickness, that is, thicker than the thin film layer typically deposited by sputtering, evaporation, printing or electroplating. The thin film layer is less than 2 micrometers.

All layers of the sensing catheter may be tightly adhered to the adjoining layers. One method of creating interfaces with good adhesion and good durability is the use a laminating press at high temperature and high pressure. Adhesives can be used to ensure that each layer stays applied to its neighboring layers.

The metal of which the foil is composed must be chosen carefully. In the case of an amperometric glucose sensor, the indicating electrode may be platinum, gold or carbon. Copper (which is commonly used as the foil for flexible electronic circuits), may not be suitable for use in a biosensor. Specifically, if there is concurrent physical contact between interstitial fluid, copper and platinum, a large galvanic current may occur as a result of a dissimilar metal junction. If a foil is used, titanium or gold can serve as the foil layer.

In some cases, there must be interconnect traces made of metal or conducting polymers that connect to the indicating electrode(s) and the reference electrode and terminate in a body-worn electronic sensor module which is in electrical continuity with the sensing catheter. The sensor module can contain a battery and a telemetry-enabled transceiver which transmits the electrochemical signals to a personal computer, mobile phone, or dedicated screen.

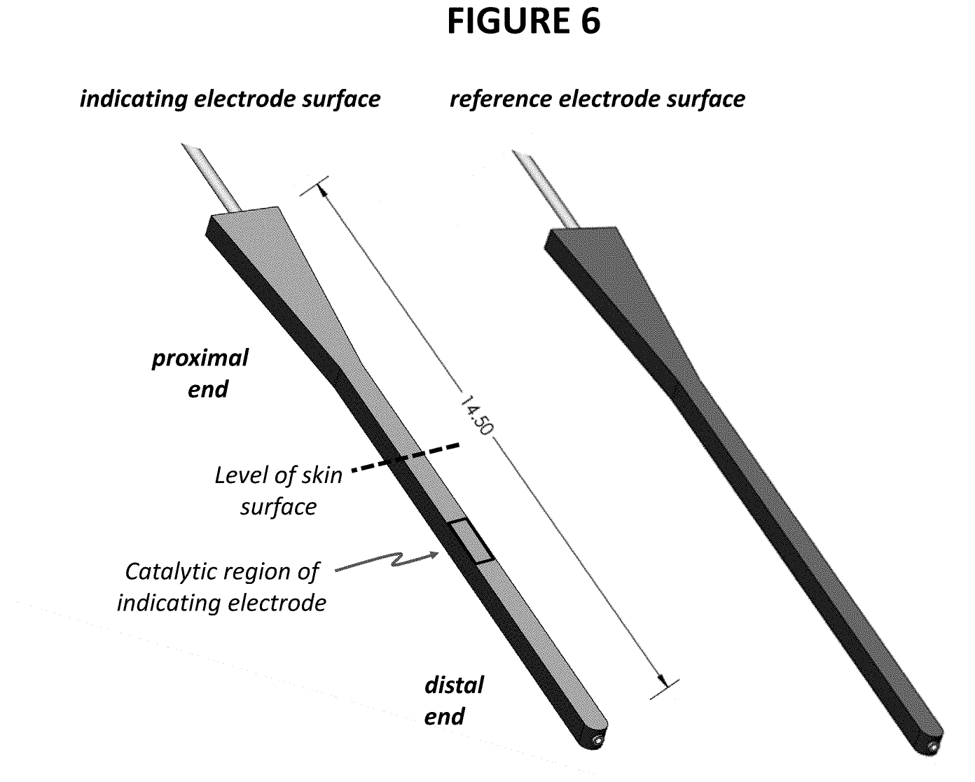

FIG. 6 illustrates an example of an embodiment of a combination sensor and cannula.

On the left is shown the indicating electrode surface of the sensing cannula, which is approximately 14.5 mm in total length, excluding the proximal extension of the fluid path tube, which can be seen to extend proximally beyond the limit of the indicating electrode. Note also that there is an inner tube that extends through the length of the device, allowing an insulin formulation to be delivered subcutaneously at the distal end. The location of the line which divides the above-skin part from the below-skin part is given by the dashed line. Typically, the skin surface is about 7 mm from the distal end of the device. In some embodiments, the surface of the indicating electrode is gold. On the right is shown the opposite surface, which is the reference electrode surface (sometimes, in a two electrode configuration such as this, which may be referred to as a combined reference-counter electrode). In some embodiments, this surface is a silver (Ag) layer chloridized with silver chloride (AgCl).

Insulin Pumps

The glucose-sensing cannula can be used with insulin pumps that have a polymeric tube between the pump and the cannula (e.g., a tube pump). In such a case, the glucose-sensing cannula connects to the end of the tubing that terminates near the skin of the patient. In addition, the glucose-sensing cannula can also be used in pumps that have a smaller total footprint, e.g., those that omit the tubing wherein the pump containing the insulin reservoir is placed on or near the skin, and the cannula that passes through the skin is directly attached to the pump—these devices may be referred to as patch pumps. In such cases, the cannula for the patch pump may comprise the glucose-sensing cannula described above. Suitable insulin pumps include, but are not limited to, those marketed by Medtronic, Tandem Corp, Sooil, Abbott, Thermalin, Roche, Insulet, Ypsomed, CellNovo, CeQur, Dana Diabecare, EO Flow, and others.

Advantages of Using Concentrated Insulin with an Integrated Glucose Sensor and Insulin Delivery Cannula Device

Since CSII users already may have an insulin cannula inserted under the skin, the use of a second percutaneous site may be considered to be an uncomfortable burden. For this and other reasons, many diabetic patients who use CSII do not use CGM. (Some use CGM most of the time, some use CGM on an occasional basis, and some never use CGM).

To address this inconvenience, a mediator-based glucose sensor may be placed in or on the outer wall of an insulin delivery cannula, as described by, for example, U.S. Pat. No. 10,780,222 (Ward et al), which is incorporated by reference herein in its entirety. For example, some of the materials and attributes of this sensing cannula, in vivo testing in swine, and in vitro tests of the device are described by (Ward, Heinrich, Breen et al, Diabetes Technol Ther 19:226-236, 2017), which is incorporated by reference herein in its entirety. By including an osmium complex (which includes an osmium-based mediator and a pyridine- or imidazole-based ligand), glucose oxidase, and a potential bias of less than 175 mV, this sensor may function in the direct presence of insulin and insulin analogs. However, when a conventional glucose sensor (requiring oxygen uptake and measuring the oxidation of hydrogen peroxide as the endpoint) was used in vitro and in vivo, there are two large types of artifacts observed. First, there is an immediate large rise in current, and second, there was a complete and permanent decay of the sensor's amperometric signal. This signal decay continues until the sensor was not even able to respond to a substantial concentration of hydrogen peroxide. Further testing indicates that these two artifacts were not caused by the insulin or insulin analog in the formulation. Instead, the artifacts are caused by the preservatives present in all insulin formulations, including phenol and a similar compound closely related to phenol, meta-cresol.

This sensing cannula may be used by patients with Type 1 diabetes. Each subject may undergo placement of two mediator-based glucose sensing cannulae placed subcutaneously in the abdomen. Through one cannula, aspart insulin (in the usual U100 concentration) may be given by a commercial insulin pump both in the basal state and at mealtimes over a one day period. Through the other sensing cannula, saline may be given at the same times and in the same volumes.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

1.-76. (canceled)

77. A system for managing blood glucose in a subject, comprising:
an insulin delivery cannula comprising a hollow tube comprising a proximal end and a distal end, wherein the proximal end is in fluid communication with a source of a concentrated insulin or insulin analog formulation, wherein the concentrated insulin or insulin analog formulation has a concentration of at least about 150 units per milliliter (units/mL), wherein the distal end is configured to deliver the concentrated insulin or insulin analog formulation into a subcutaneous space of the subject;
a glucose sensor located no more than a pre-determined distance away from the distal end, wherein the glucose sensor is a continuous amperometric or coulometric glucose sensor comprising at least one indicating electrode; and
an insulin pump fluidically coupled to the proximal end of the insulin delivery cannula, wherein the insulin pump is attached to the insulin delivery cannula either directly or via an intervening tube,
wherein the glucose sensor and the insulin delivery cannula are configured to be inserted into the subcutaneous space of the subject simultaneously by a single insertion device.

78. The system of claim 77, wherein the concentrated insulin or insulin analog formulation comprises an excipient comprising a phenolic compound or cresol.

79. The system of claim 77, wherein the pre-determined distance is about 7 mm.

80. The system of claim 77, wherein the glucose sensor comprises an electrode layer comprising the at least one indicating electrode, wherein the electrode layer underlies a redox-catalytic layer comprising (1) a redox mediator comprising a metal compound covalently bound to a ligand, and (2) an enzyme comprising glucose oxidase or glucose dehydrogenase.

81. The system of claim 80, wherein the glucose sensor further comprises an insulating layer and a metal layer, wherein the insulating layer is coupled to the metal layer, and wherein the metal layer is coupled to the electrode layer.

82. The system of claim 80, wherein the redox mediator and the enzyme allow electron transfer from subcutaneous glucose to the at least one indicating electrode sufficient to cause a response of the glucose sensor to a subcutaneous glucose concentration of the subject at an applied bias potential of no more than about +300 millivolts (mV), +250 mV, +200 mV, +150 mV, +100 mV, or +50 mV relative to a reference electrode.

83. The system of claim 82, wherein the applied bias potential of no more than about +250 mV, +200 mV, +150 mV, +100 mV, or +50 mV relative to the reference electrode allows the electrode layer to undergo substantially no electropolymerization of the excipient during continuous operation of at least one hour of the amperometric glucose sensor, thereby maintaining a sensitivity of the amperometric glucose sensor to the subcutaneous glucose concentration in presence of the concentrated insulin or insulin analog formulation.

84. The system of claim 80, wherein the metal compound comprises a metal selected from the group consisting of: Osmium, Ruthenium, Palladium, Platinum, Rhodium, Iridium, Cobalt, Iron, and Copper.

85. The system of claim 77, wherein the glucose sensor is disposed on an outer wall of the hollow tube.

86. The system of claim 77, wherein the insulin pump is a tube pump.

87. The system of claim 77, wherein the insulin pump is a patch pump.

88. A method for managing blood glucose in a subject, comprising:
(a) obtaining a device for delivery of the insulin or insulin analog formulation and measurement of subcutaneous glucose concentration, wherein the device comprises:
(i) a hollow tube comprising a proximal end and a distal end, wherein the proximal end is in fluid communication with a source of the concentrated insulin or insulin analog formulation, wherein the concentrated insulin or insulin analog formulation has a concentration of at least about 150 units per milliliter (units/mL), wherein the distal end is configured to deliver the concentrated insulin or insulin analog formulation into a subcutaneous space of the subject; and
(ii) a glucose sensor located no more than a pre-determined distance away from the distal end, wherein the glucose sensor is a continuous amperometric or coulometric glucose sensor comprising at least one indicating electrode;
(b) connecting the proximal end of the hollow tube to the source of the concentrated insulin or insulin analog formulation;
(c) inserting the glucose sensor and the distal end of the hollow tube of the insulin delivery cannula into the subcutaneous space of the subject simultaneously by a single insertion device; and
(d) simultaneously (1) delivering the concentrated insulin or insulin analog formulation into a subcutaneous space of the subject using an insulin pump fluidically coupled to the proximal end of the insulin delivery cannula, wherein the insulin pump is attached to the insulin delivery cannula either directly or via an intervening tube, and (2) measuring the subcutaneous glucose concentration of the subject.

89. The method of claim 88, wherein the concentrated insulin or insulin analog formulation comprises an excipient comprising a phenolic compound or cresol.

90. The method of claim 88, wherein the pre-determined distance is about 7 mm.

91. The method of claim 88, wherein the glucose sensor comprises an electrode layer comprising the at least one indicating electrode, wherein the electrode layer underlies a redox-catalytic layer comprising (1) a redox mediator comprising a metal compound covalently bound to a ligand, and (2) an enzyme comprising glucose oxidase or glucose dehydrogenase.

92. The method of claim 91, wherein the glucose sensor further comprises an insulating layer and a metal layer, wherein the insulating layer is coupled to the metal layer, and wherein the metal layer is coupled to the electrode layer.

93. The method of claim 91, wherein the redox mediator and the enzyme allow electron transfer from subcutaneous glucose to the at least one indicating electrode sufficient to cause a response of the glucose sensor to a subcutaneous glucose concentration of the subject at an applied bias potential of no more than about +300 millivolts (mV), +250 mV, +200 mV, +150 mV, +100 mV, or +50 mV relative to a reference electrode.

94. The method of claim 93, wherein the applied bias potential of no more than about +250 mV, +200 mV, +150 mV, +100 mV, or +50 mV relative to the reference electrode allows the electrode layer to undergo substantially no electropolymerization of the excipient during continuous operation of at least one hour of the amperometric glucose sensor, thereby maintaining a sensitivity of the amperometric glucose sensor to the subcutaneous glucose concentration in presence of the concentrated insulin or insulin analog formulation.

95. The method of claim 91, wherein the metal compound comprises a metal selected from the group consisting of: Osmium, Ruthenium, Palladium, Platinum, Rhodium, Iridium, Cobalt, Iron, and Copper.

96. The method of claim 88, wherein the glucose sensor is disposed on an outer wall of the hollow tube.

97. The method of claim 88, wherein the insulin pump is a tube pump.

98. The method of claim 88, wherein the insulin pump is a patch pump.
